(19) United States
(12) Patent Application Publication
(10) Pub. No.: US 2002/0026209 A1
(43) Pub. Date: Feb. 28, 2002

(54) Method and device for obtaining prostatic material

(76) Inventor: David Hung, Belmont, CA (US)

Correspondence Address: BANNER & WITCOFF, 1001 G STREET N W, SUITE 1100, WASHINGTON, DC, 20001, US

(21) Appl. No.: 09/912,055

(22) Filed: Jul. 25, 2001

Related U.S. Application Data
(60) Provisional application No. 60/220,799, filed on Jul. 25, 2000.

(51) Int. Cl. A61M 1/00

(52) U.S. Cl. 606/192; 604/540; 604/27

(57) ABSTRACT

A method and apparatus for obtaining prostatic material by introducing a washing fluid to a prostatic portion of a urethra and collecting at least a portion of the washing fluid comprising prostatic material for analysis.

### CROSS REFERENCE TO RELATED APPLICATIONS

This application claims the benefit under 37 CFR 1.78 of provisional application No. 60/220,799 filed on Jul. 25, 2000 and provisional application No. 60/249,275 filed Nov. 17, 2000. The full disclosures of each of the prior applications are incorporated herein by reference.

### FIELD OF THE INVENTION

The present invention includes a method and device for obtaining material from a body of a patient. More specifically, the present invention includes a method and device introducing fluid into and for obtaining prostatic material from a patient.

### BACKGROUND OF THE INVENTION

The prostate gland is a partly glandular and partly muscular body about the size of a chestnut located immediately below the neck of the bladder and around the commencement of the urethra. The prostate can have a volume between 20 cc and 60 cc. It is also possible to have a volume that is less than 20 cc or greater than 60 cc. The glandular substance is composed of numerous follicular pouches opening into elongated canals, which join to form from approximately 12 to 20 small secretory ducts. The follicles are connected together by areolar tissue, supported by prolongation from the fibrous capsule and muscular stroma, and enclosed in a delicate capillary plexus. The epithelium lining of both the canals (secretory ducts) and the terminal vesicles is of the columnar variety. The prostatic secretory ducts (approximately 12-20 of them) open into the floor of the prostatic portion of the urethra. Gray's Anatomy, p. 1009-1010 Running Press Edition, Phil., Pa. 1974.

Prostate cancer is the second most common cancer in men with 130,000 cases diagnosed and about 34,000 deaths occurring each year in the United States alone. Prostate cancer is detected presently by urinary dysfunction, palpation in a rectal exam, or elevated levels of prostate specific antigen (PSA) in the blood. Treatment options for men diagnosed with prostate-confined adenocarcinoma include observation, radical prostatectomy, external beam radiotherapy, radioactive prostate implantation, hormonal therapy, cryotherapy, and various combinations of these treatments. Side effects of these treatments can be loss of sexual function and/or urinary incontinence. It would be advantageous to the medical community to provide alternative methods and devices in order to diagnose and treat prostate cancer, and other conditions of the prostate.

### SUMMARY OF THE INVENTION

An aspect of the present invention comprises a method for obtaining prostatic material from a patient that after undergoing a cytological analysis will provide information sufficient to identify a precancerous or cancerous condition in the prostate. One embodiment of the present invention includes a method for collecting prostatic material. The method also includes the steps of introducing a washing fluid to a prostatic portion of a urethra and collecting at least a portion of the washing fluid comprising prostatic material.

In another aspect, the present invention includes a method for collecting material from one or more prostatic ducts of a patient. This method includes the steps of introducing a device through a urethra of a patient, locating the device in or near a prostatic portion of said urethra, and introducing a washing fluid throughout the prostatic portion. The method also includes the step of collecting at least a portion of the washing fluid mixed with prostatic material.

An aspect of the present invention includes a method for collecting prostatic material. The method comprises the steps of introducing a washing fluid to a prostatic portion of a urethra and collecting at least a portion of the washing fluid comprising the prostatic material. The methods of the present invention can be used to collect material from one or more prostatic ducts of a patient. The steps comprise introducing a device through a urethra of a patient, locating the device in or near a prostatic portion of the urethra, introducing a washing fluid throughout the prostatic portion, and collecting at least a portion of the washing fluid mixed with prostatic material.

An aspect of the present invention also includes a method of treating a prostate condition in a patient comprising the steps of locating a prostatic portion of a urethra, and introducing a composition comprising a prophylactic or therapeutic agent to the prostatic portion. Another aspect of the invention comprises a method of treating a prostate condition in a patient. This method comprises the steps of accessing a prostatic portion of a urethra with an access device and introducing a composition comprising a prophylactic or therapeutic agent to the prostatic portion.

A device according to an aspect of the present invention can access a prostatic portion of a urethra. The device comprises an access lumen to infuse wash fluid into the prostatic portion of the urethra and collect prostatic material mixed with wash fluid from within the prostatic portion of the urethra.

A device according to another aspect of the invention can also access a prostatic portion of a urethra. This device comprises at least one lumen, a first expandable positioning member and a second expandable positioning member. The device also comprises a fluid delivery portion located between the expandable positioning members for being located proximate the prostatic ducts within the urethra. The fluid delivery portion includes at least one port for infusing fluid into at least one of the prostatic ducts after the expandable positioning members have been expanded.

A device for accessing a prostatic portion of a urethra according to a further aspect of the invention comprises at least one lumen and an expandable fluid delivery portion for being positioned proximate the prostatic ducts within the urethra. The fluid delivery portion includes at least one port for infusing fluid into at least one of the prostatic ducts after being expanded.

A device for accessing a prostatic portion of a urethra according to another aspect of the invention comprises at least one lumen having a diameter that tapers over at least a portion of its length in a direction of a distal end. This taper permits a fluid delivery portion of the device to form a tight fit with an inner wall of the urethra that carries a plurality of prostatic ducts. The fluid delivery portion includes at least one fluid delivery port for infusing fluid into at least one of the prostatic ducts.

The present invention also includes a device for accessing a prostatic portion of a urethra comprising at least one lumen having a flexible distal end that includes an infusion port, a fiberoptic scope and a guidewire formed of a shape memory material. When a stimulus is applied to the guidewire, the guidewire alters an orientation of the flexible distal end of the lumen A further aspect of the present invention comprises a system for obtaining material from a secretory duct of a prostate. The system comprises a device comprising a lumen for accessing a prostatic portion of a urethra and introducing fluid into that portion and any secretory ducts that open into the prostatic portion of the urethra; instructions for using the device as described in the methods herein, and a container for the system contents. The system can also comprise reagents for washing or collecting the wash fluid; and reagents for preservation or analysis of the washing fluid collected from one or more of the prostatic secretory ducts. The device may have at least one lumen, and more if necessary for its function to introduce washing fluid into the prostatic portion of the urethra and collect the introduced fluid mixed with secretory ductal fluid. The reagents for washing can include a biocompatible solution such as, for example, saline or PBS. The reagents for collection may include reagents that increase the material collected, such as oncotic or osmotic agents that draw fluid to them from the prostate gland. Reagents for preservation can include, e.g., reagents to preserve the epithelial cells collected and/or the markers that will be identified. Reagents for analysis of the material collected can include, e.g., stain for the epithelial cells, or agents to identify the markers sought (e.g., antibodies, or other tags or labels).

According to an additional aspect of the present invention, a device comprising a tube or lumen may be introduced into the penile urethra and encouraged to a position of proximity to the prostatic portion of the urethra. A washing fluid may travel into the tube or lumen to contact and/or occupy the prostatic portion of the urethra. It may be useful to block this fluid receiving area in the prostatic portion of the urethra with stops on the access device, so that the prostatic portion can fill with fluid without losing the fluid to other regions of the urethra. Thus, one embodiment of the invention employs an access device comprising a lumen capable of accessing the urethra and being guided to the prostatic portion, or into proximity with the prostatic portion of the urethra sufficient for fluid introduction into the region in order to provide wash fluid the opportunity to enter into one or more of the prostatic secretory ducts. The position of the access device maybe determined to be at a correct position for accomplishing the task at hand by a number of methods, including, e.g., the length of tubing fed into the urethra, and/or a rectal palpation, ultrasound or magnetic resonance imaging (MRI) to determine that the tip is at the prostatic portion.

### BRIEF DESCRIPTION OF THE FIGURES

FIG. 1 depicts relevant male anatomy including bladder, urethra and prostate;

FIG. 2 depicts a device accessing the prostatic portion of the urethra;

FIG. 3 depicts access of the prostatic portion of the urethra by a device according to another embodiment of the present invention;

FIG. 4 depicts an access device according to the present invention comprising positioning devices;

FIGS. 5 and 6 depict the access device of FIG. 4 with the positioning devices located in the urethra on either side of the prostatic ducts and infusing fluid into the ducts;

FIG. 7 depicts another embodiment of the access device according to the present invention;

FIG. 8 depicts the expanded access device of FIG. 7 positioned within the urethra in close contact with the prostatic ducts;

FIG. 9 depicts another access device according to the present invention including a fiberoptic scope and a guidewire; and

FIGS. 10 and 11 illustrate the access device of FIG. 9 with a distal end of the catheter and guidewire in a ductal access/contact position.

### DESCRIPTION OF THE SPECIFIC EMBODIMENTS

The following preferred embodiments and examples are offered by way of illustration and not by way of limitation.

FIG. 1 illustrates relevant portions of the male anatomy including the bladder 10, the urethra 11 and the prostate 15. As is well known, the bladder 10 is connected to one end of the urethra 11. Hence, fluid exiting the bladder 10 flows past a neck of the bladder 10 and into the urethra 11. As this fluid flows through the urethra 11 and away from the bladder 10, it passes along a portion 13 of urethra 11 that is surrounded by the prostate 15 and through the prostatic portion of the urethra 14. The fluid is eventually voided through the urethra opening 12.

FIG. 2 depicts an embodiment of a device 100 including a catheter 17 with a longitudinally extending lumen. The device 100 also includes an infusion syringe 18 and a distal tip 16. The catheter 17 accesses the urethra 11 through orifice 12 and extends to the prostatic portion of the urethra 14. The device 100 delivers a wash fluid from the infusion syringe 18, through catheter 17, past distal tip 16. After the washing fluid mixes with material from the secretory ducts that open into the prostatic portion of the urethra 14, the washing fluid and prostatic material can be collected by device 100 through distal tip 16.

In some instances, the distal tip 16 of the device 100 can extend beyond the prostatic portion of the urethra 14. When this occurs, the fluid that exits the distal tip 16 should be allowed to make contact with the prostatic portion of the urethra 14. After this contact is made, the induced wash fluid and prostatic material can be collected from the prostatic portion of the urethra 14.

Alternatively, in an embodiment of device 100, the catheter 17 can include a port located at a position along its length that permits the wash fluid to mix with the prostatic material without having to exit distal tip 16. The port is positioned so that the fluids exiting the device 100 through it make contact with the prostatic portion of the urethra 14 even though the distal tip 16 is positioned beyond this portion of the urethra 11.

FIG. 3 depicts an embodiment of the device 100 that accesses the penile urethra 11 through opening 12. The device 100 includes a single lumen catheter 17 that extends into and along the urethra 11 so that the distal tip 16 of device 100 is positioned in the prostatic portion of the urethra 14. The device 100 also includes an infusion lumen 27 that is in fluid communication with, and extends away from, the lumen 17. An infusion syringe 18 for introducing fluid into the infusion lumen 27 and the single lumen catheter 17 is connected to the infusion lumen 27 as shown in FIG. 3. A collection lumen 19 is in fluid communication with the single lumen catheter 17. Like infusion lumen 27, collection lumen 19 extends away from the single lumen catheter 17. The end of collection lumen 19 distal the catheter 17 is in fluid communication with a collection receptacle 20 for collecting wash fluid comprising prostatic material after the wash fluid has been introduced into the body and contacts the prostatic secretory ducts.

The access device 100 according to the present invention may have one or more lumens. For example, the device 100 can include a first lumen for fluid infusion, and a second lumen for fluid collection. Also, for example, the device 100 can comprise in addition to a first lumen (or a first and second lumen) a stiffening wire or tube. A visualizing device (e.g., a scope or other device) might be used in the access device 100, to position the distal tip 16 of the device 100 in the prostatic portion of the urethra 14. Another embodiment includes a device that is placed over the opening 12 of the urethra 11 and is capable of introducing fluid into the urethra 11 in a retrograde fashion to voiding, without deep penetration of the urethra 11. This embodiment provides for introduction of fluid into the prostatic portion of the urethra 14 and one or more of the secretory ducts that empty in that portion 14. At least a portion of the fluid so introduced is collected, comprising prostatic material.

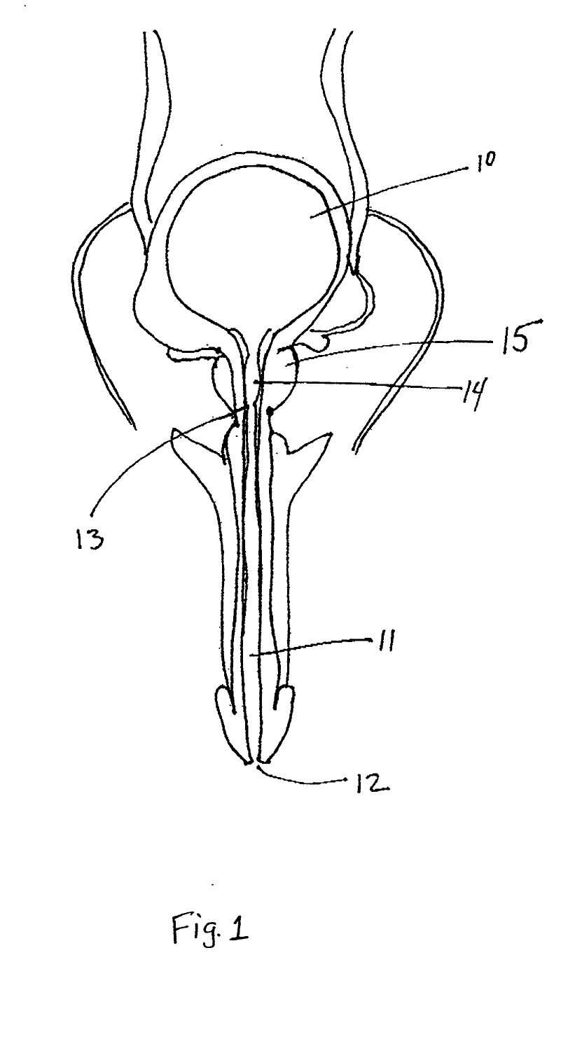

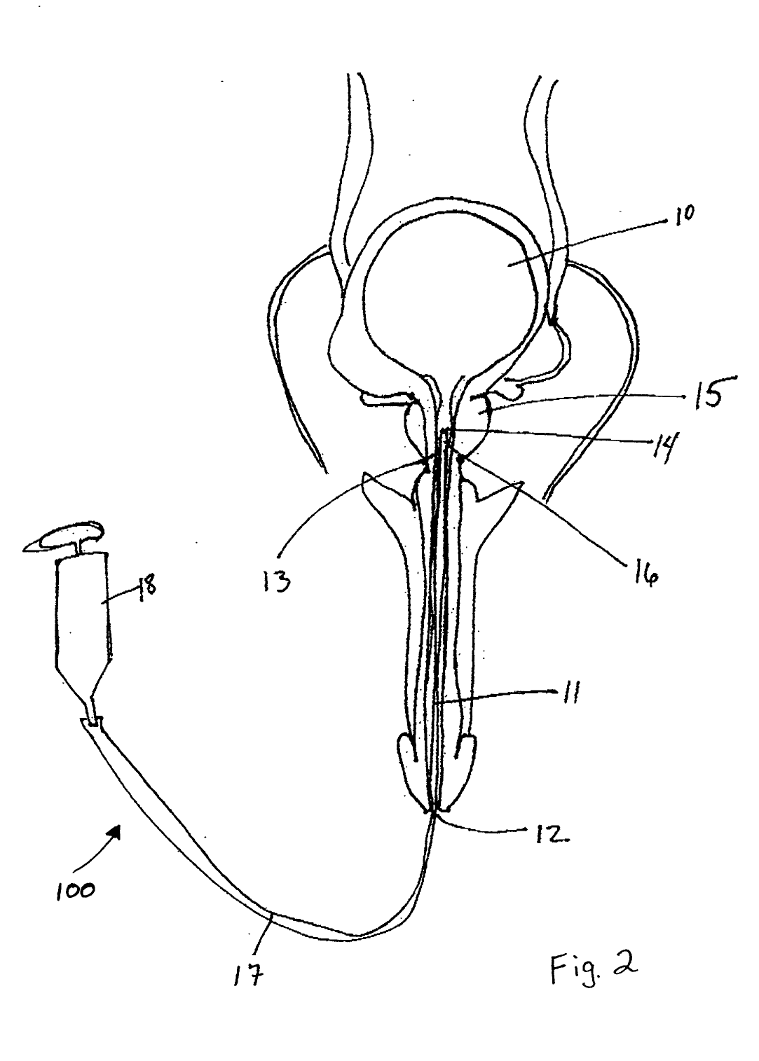

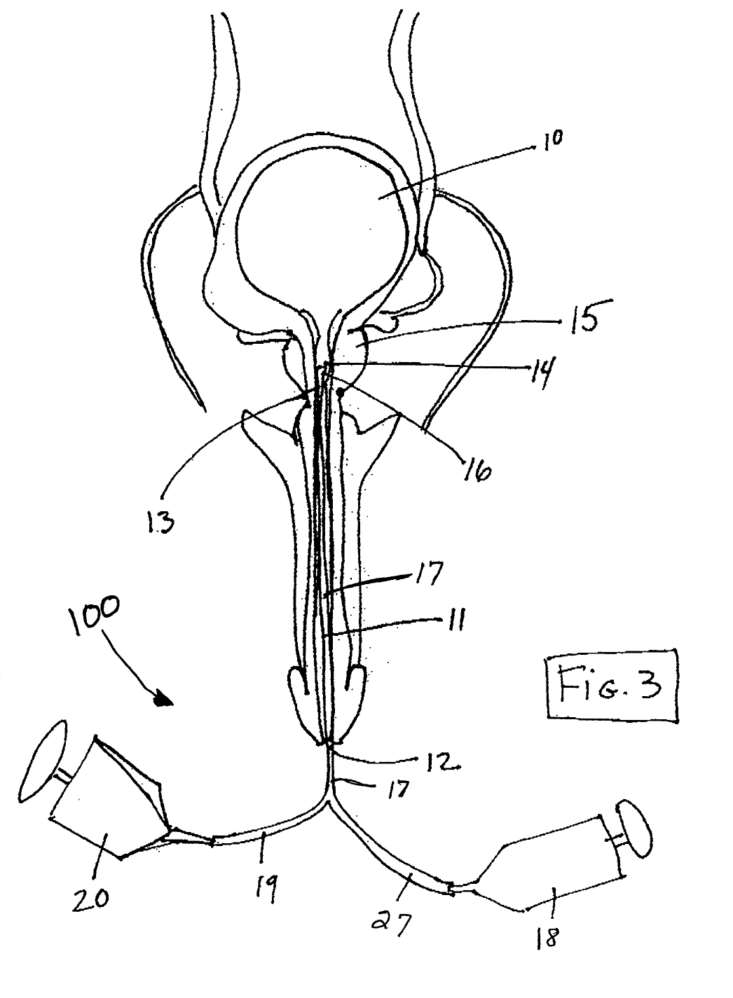

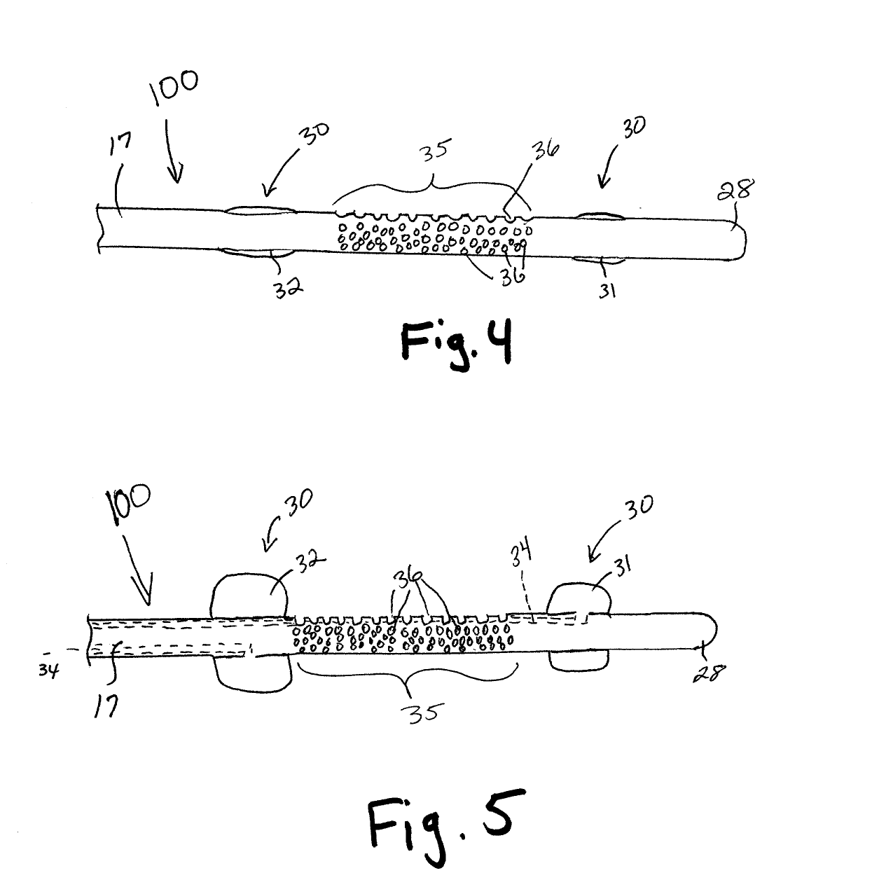

In another embodiment according to the present invention, illustrated in FIG. 4, the access device 100 includes at least one positioning device 30 used to position and locate at least a portion of the device 100 at a predetermined location within the prostatic portion 14 so that fluid can be introduced into the prostatic ducts 85 within the prostate 15. As shown in FIGS. 4 and 5, the catheter 17 includes a first positioning device 31 proximate the distal end 28 of the catheter 17 and a second positioning device 32 positioned on an opposite side of fluid delivery section 35 and proximate the proximate end of the catheter 17. The positioning devices 31, 32 are spaced from each other along the length of the catheter 17 by a distance equal to the length of a prostate. This actual distance can change from patient to patient. However, it is approximately the length of a chestnut. It is also possible that the positioning devices 31, 32 be slidable along the length of the catheter 17 so the space between them can be adjusted to meet the size of particular patient.

In one embodiment, the positioning devices are expandable and can include inflatable members 33, such as balloons. Each inflatable member 33 is in fluid communication with an inflation lumen 34 that extends to the proximal end of the access device 100 as shown in phantom lines in FIG. 5. The inflatable members 33 can be in fluid communication with the same inflation lumen 34 or separate inflation lumens 34, 34. Other known ways of expanding a positioning member 30 can also be used. These other known ways can include structures formed of shape memory materials and structures that can be mechanically manipulated into an expanded form.

The fluid delivery section 35 extending between the two positioning devices 30 includes at least one port 36 for infusing fluid into the prostatic ducts 85. In one embodiment, as shown in FIGS. 4 and 5, the fluid delivery section 35 includes a plurality of infusion ports 36 spaced around the circumference and along the length of the catheter 17. These ports 36 deliver fluid from within the catheter 17 to the prostatic ducts 85.

During positioning of device 100, the catheter 17 is introduced within the prostatic portion of the urethra 14 until the ports 36 in the fluid delivery section 35 are positioned in the same location as the prostatic ducts 85. Determining the position of the fluid delivery section 35 relative to the ducts 85 can be accomplished manually by measuring the patient and inserting the catheter 17 into the patient to a depth that accounts for the measured distance. Additionally, it may be accomplished by introducing the catheter 17 into the prostatic portion of the urethra 14 until resistance to the movement of the catheter 17 within the patient is encountered. Positioning the catheter 17 can also be manually accomplished by rectal examination - feeling the position of the catheter 17 through the rectum. Moreover, positioning the catheter 17 can also be accomplished by imaging or other known visual techniques. External imaging can include the use of known external imaging sources such as ultrasound, X-ray, MRI or other imaging techniques. An internally positioned fiberoptic scope, such as a conventional fiberoptic endoscope, can also be used to determine the location of the ducts 85 and the relative position of the fluid delivery section 35 to the ducts 85. When a fiberoptic scope is used, the distance between the distal end of the scope and the fluid delivery section 35, if any, can be added on to the introduction of the catheter 17 after the last duct to receive the fluid has been located.

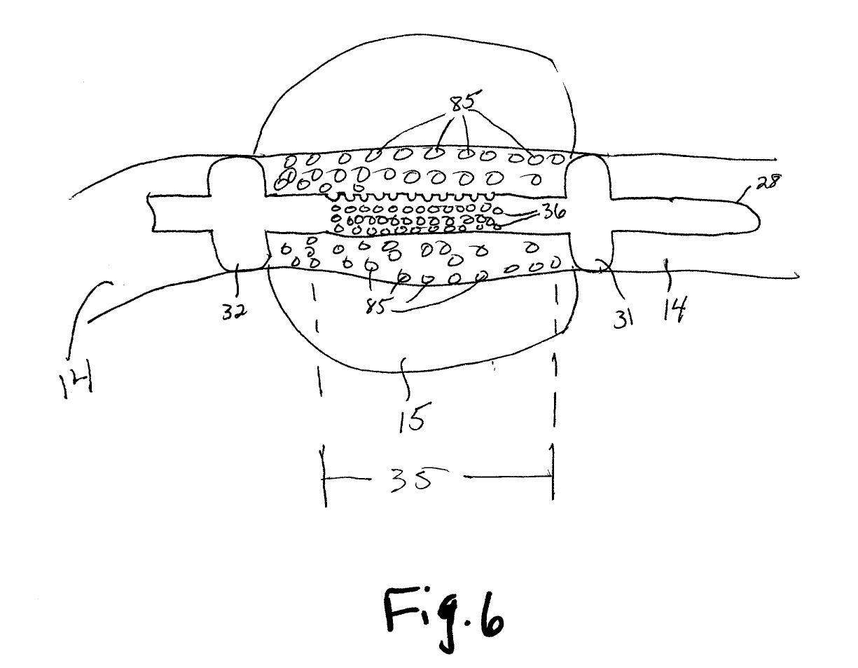

After the fluid delivery section 35 has been positioned at the prostatic ducts 85, shown in FIG. 6, the positioning devices 31, 32 are expanded. After their expansion, the positioning devices 31, 32 not only hold the fluid delivery section 3 5 in place, but they also act as barriers (stops) to fluid flowing into or out of the section of the urethra blocked by the positioning devices 31, 32. As a result, when fluid is delivered under pressure to the port(s) 36 of the fluid delivery section 35 through catheter 17, the fluid pressure within the prostatic portion of the urethra 14 between the positioning devices 31, 32 is greater than the resistance provided by the prostatic ducts 85 and the fluid from the port(s) 36 is infused under pressure into the prostatic ducts 85.

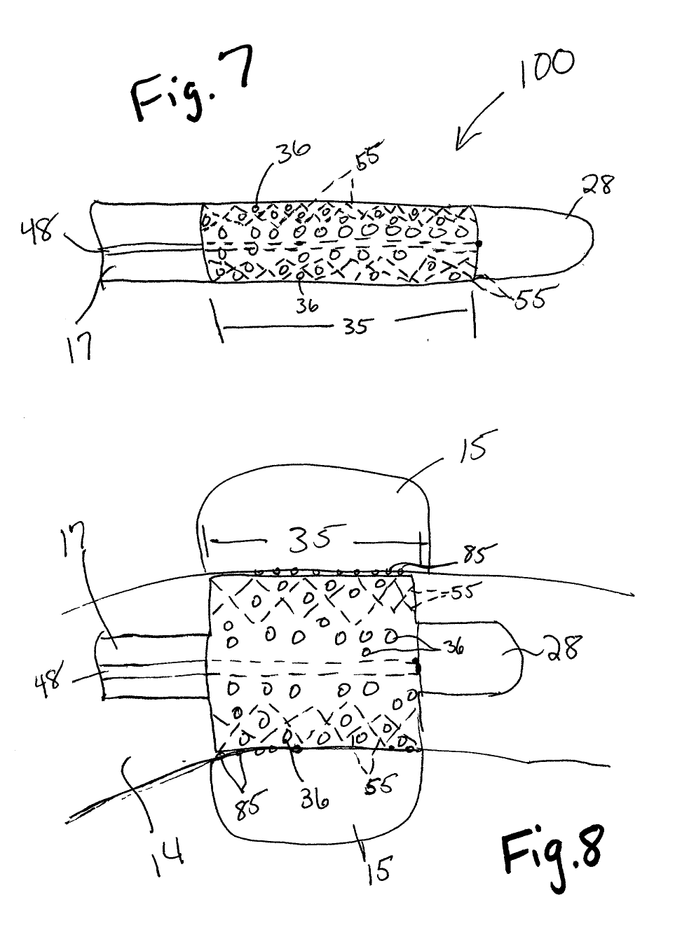

In an alternative embodiment illustrated in FIG. 7, the fluid delivery section 35 is formed on an expandable portion of catheter 17 so that a contact or substantially in contact fit is achieved between section 35 and the inner walls of the prostatic portion of the urethra 14 carrying the prostatic ducts 85. In this embodiment, the portion of catheter 17 carrying the fluid delivery section 35 can include a structure 55 (shown in broken) that expands in response to mechanical manipulation or application of a stimulus so that a snug contact or substantially in contact fit is achieved between the fluid delivery section 35 and at least the wall of the prostatic portion of the urethra 14 carrying the prostatic ducts 85. For example, as shown in FIG. 8, this portion of the catheter 17 can include a structure that will expand when a longitudinally compressive force is applied to it. A wire or other elongated member 48 secured to one end of the catheter 17 and extending through the length of the catheter 17 so that it can be pulled when expansion is desired can apply such a force. Such an arrangement is used in conventional stents.

In an alternative embodiment, this expandable fluid delivery section 35 of the catheter can be formed of a shaped memory material that will take an expanded shape in response to the application of a stimulus such as heat or electrical current that is delivered in a conventional manner. Additionally, the lumen of the catheter 17 can be tapered in the direction of its distal end so that the fluid delivery section 35 has substantially the same diameter as the prostatic portion of the urethra 14 and a snug fit is achieved between the fluid delivery section 35 and the prostatic portion of the urethra 14 carrying the prostatic ducts 85. In any of the above-discussed embodiments, the snug, close fit between the fluid delivery section 35 and the ducts allows fluid delivered to the port(s) 36 to be infused under pressure into the prostatic ducts 85. The diameter of the expanded potion of the catheter 17, the portion that forms a snug fit with at least the prostatic portion of the urethra 14 is from 1 mm to 10 mm. A preferred diameter is from 4 mm to 6 mm.

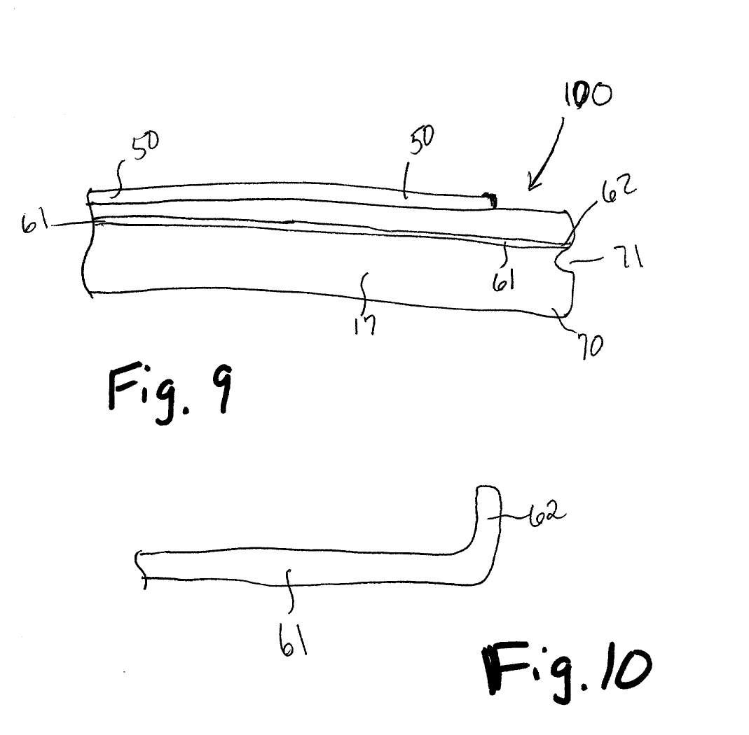

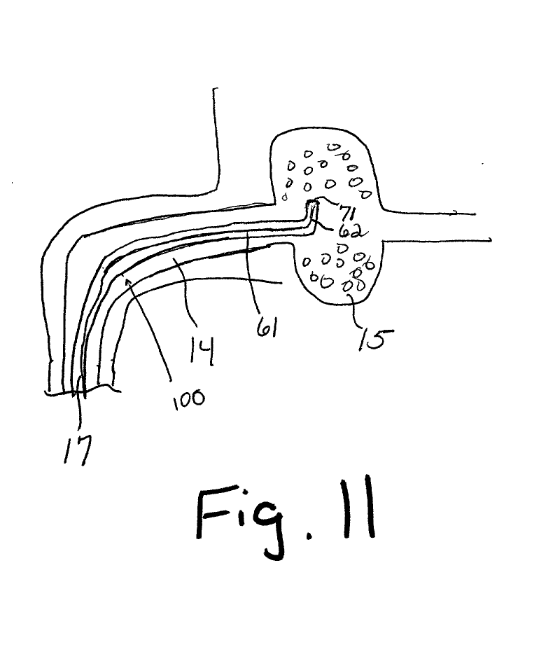

As shown in FIG. 9, the catheter 17 can include a fiber optic scope 50 used to locate the prostatic ducts 85. The catheter 17 also includes a guidewire 61 formed of a shape memory material that is positioned so that its distal end 62 is proximate a flexible distal end of the catheter 17. During introduction of the catheter 17, the guidewire 61 is straight as illustrated in FIG. 9. However, after an abnormal prostatic duct has been located, such as an enlarged duct or an abnormally shaped duct, a stimulus is applied to the guidewire 61 and its distal end 62 bends into a ductal accessing form. One example of such a form—a substantially ninety-degree bend—is illustrated in FIG. 10. The flexible distal end 70 of the catheter 17 that takes the same shape of the altered distal end 62 of the guidewire 61 is then moved in the direction of the abnormal or chosen duct. In this embodiment, a fluid delivery port 71 of the catheter 17 can access or be steered directly against the opening of a prostatic duct for infusing a wash fluid into the duct, as shown in FIG. 11. More than one duct can be accessed if more than one lumen is included in the device 10 and the lumens are separately rotatable.

As used herein, “shape memory” materials include any known material that returns to a predetermined shape or position in response to the application of a stimulus, such as heat, cold or electrical current. Well-known shape memory materials include shape memory alloys such as Nitinol. Shape memory materials that exhibit super elastic properties, such as superelastic Nitinol, may also be used.

As discussed above, an aspect of the present invention includes methods for introducing a wash fluid into the urethra 11 and collecting prostatic material from the prostate. These methods can be accomplished by introducing a washing fluid throughout the prostatic portion of a urethra 14 using the access device 100. Alternatively, any tool capable of accessing and introducing a fluid into the prostatic portion of the urethra 14 through the urethra opening 12 could also be used. Although the invention is not limited by theories of how the invention might work, it may be that by introducing washing fluid throughout the prostatic portion of the urethra 14, at least some of the washing fluid introduced will flow or otherwise find its way into at least a part of one or more prostatic secretory ducts that open into the prostatic portion of the urethra 14. It may be sufficient to simply introduce fluid into this portion 14, and it may also be beneficial to fill the portion 14 to the point at which the fluid is encouraged to enter the prostatic secretory ducts that open to the prostatic portion of the urethra 14. The fluid can be introduced by the device 100 including catheter 17 or by any other device believed effective for delivering fluid through the urethra 11, provided that the fluid introduced reaches and/or contacts the prostatic portion of the urethra 14.

Collecting a portion of the wash fluid introduced into the urethra 11 can be accomplished using the lumen or tube 17 that can receive the wash fluid comprising prostatic material. The wash fluid comprising prostatic material can also be encouraged to travel in the lumen or tube 17 to a collecting receptacle such as syringe 18 (shown in FIG. 2) or collection receptacle 20 (shown in FIG. 3). According to the method, enough wash fluid can be introduced into the prostatic portion of the urethra 14 to provide a negative back pressure that can encourage the wash fluid mixed with prostatic material to travel back into the device 100 and into one of the above-mentioned collection receptacles. A lavage, or washing fluid dynamic can be established in the urethra 11, and/or in the prostatic portion of the urethra 14, so that wash fluid is introduced, and this fluid flushes the region including portions of one or more prostatic secretory ducts. The wash fluid mixed with prostatic material is then collected.

The volume of washing fluid introduced for a given collection procedure may be in a range from about 0.1 cc of fluid to about 100 cc of fluid, depending upon such variables, for example, as the size and/or expandability of the prostatic portion or the urethra, and/or the number of secretory ducts that receive fluid, the extent that the spaces accessed or contacted can accommodate fluid that is introduced, and the number of times that the wash fluid is introduced and collected. In some cases, for example, from about 0.5 cc to about 20 cc of wash fluid can be introduced at a time. Fluid can be introduced in multiple intervals, and then also collected in subsequent intervals. For example 2 ccs may be introduced, followed by another 2 ccs a time interval later. From the same perspective, fluid can be introduced and collected and then new fluid can be introduced, and a second aliquot collected, etc. For example 2.5 ccs of fluid can be introduced, and a time interval later a first aliquot can be collected; then another 2.5 ccs of wash fluid can be introduced and a time interval later a second aliquot can be collected.

The washing fluid is preferably a biocompatable fluid having an isotonic composition and/or a biological pH. The wash fluid can comprise such as for example saline or phosphate buffered saline (PBS). Any wash fluid suitable for introduction to humans can be used.

The prostatic material collected can comprise cellular species. The cellular species can comprise prostatic ductal epithelial cells. Prostatic ductal epithelial cells are important because they may indicate a hyperplastic, atypical or carcinoma in situ type condition that would indicate a pre-cancer in the first two instances, and a pre-invasive cancer in the latter instance. Prostatic ductal epithelial cells may also be analyzed to reveal conditions other than cancer or pre-cancer. Analysis of any cells collected can be made by cytology.

Identification of the prostatic epithelial cells comprises separating the cells in the collected fluid by standard cell separation techniques (e.g. centrifugation). The separated cells may be preserved and/or fixed onto a solid substrate (such a glass slide) for viewing under a microscope. The cells can be examined for their morphology or other indicia of abnormality. Abnormal cells can be, for example, hyperplastic, atypical, neoplastic, malignant, etc. Atypical cells may assess the likelihood of cancer or precancer present in the lining of the secretory duct from which the cells were collected. It is believed that high-grade prostatic intraepithelial neoplasia (PIN) is most likely the preinvasive stage of adenocarcinoma in the prostate. See, Bostwick Semin Urol. Oncol. 1999; 17(4): 187-98. Thus PIN is regarded as a highly predictive marker of adenocarcinoma. Cytological analysis of prostatic secretory duct cells may provide information sufficient to identify a condition of precancer or cancer in the prostate. Cytology has been applied to cells derived from the breast, lung, colon and other tissues to identify conditions that are characterized by cellular abnormality, such as cancer. Cytological analysis of secretory ductal epithelial cells from the prostate may detect normal cells, hyperplastic cells, low-grade prostatic intraepithelial neoplasia, high-grade PIN, carcinoma and invasive carcinoma. Cytological analysis may be conducted as is standard in the art, for example, applying epithelial cells to a slide, staining the cells, and viewing them under a microscope for their morphological characteristics. The washing fluid containing material from prostatic secretory ducts may be analyzed cytologically to determine the cytological status of the secretory ductal epithelial cells and other cells. The ductal fluid can be analyzed by cytological techniques by placing some of the fluid on a slide with a standard cytological stain and using a light microscope to identify the cell morphology.

In a cytological analysis, the ductal epithelial cells in the sample are evaluated for one or more indicia such as cell grouping, cell shape, cell size, nuclear size, nuclear shape, presence or absence of nucleoli, nuclear-to-cytoplasmic ratio, vacuoles in the cytoplasm, cytoplasmic shape, cytoplasmic border, presence or absence of anisonucleosis, presence or absence of mitotic figures, nuclear membrane quality, presence of necrotic debris, chromatin distribution, coarseness of chromatin, and the presence or absence of microcalcifications. Based on the presence of any one or more of the observable indicia listed, the sample is then classified as being normal (or benign), atypical (including either low-grade or high-grade prostatic intraepithelial neoplasia (PIN) or malignant (i.e. carcinoma or invasive carcinoma).

The prostatic material collected can comprise molecular species. The molecular species can be anything that is a molecule and which is collectable from a prostate as described in the method. Such molecular species are useful for diagnosing a condition of the prostate. The molecular species can include, for example, a protein, a polypeptide, a peptide, a nucleic acid, a polynucleotide, an mRNA, a small organic molecule, a lipid, a phospholipid, a fat, a glycoprotein, a glycopeptide, a carbohydrate, an oligosaccharide, a chromosomal abnormality, a particle, a secreted molecule, a cell surface molecule, an intracellular molecule and a complex of a plurality of molecules. The molecular species of interest and usefulness will generally be a species that can be detected once collected, for example using available assay and detection methods developed for that or similar or analogous purposes. Detection and analysis of these classifications of markers can be accomplished, using standard assays for determining the presence of markers or marker classifications listed; for example standard procedures for conducting such assays (e.g., ELISA, RNA or DNA probe hybridization, immunohistochemical assays, and binding or other detection assays) are described generally in Sambrook et al., Molecular Cloning: A Laboratory Manual, 2^{nd} Ed. (Cold Spring Harbor Press, Cold Spring Harbor, N.Y. 1989).

At least a portion of the wash fluid contacts a portion of one or more secretory ducts, and is then collected mixed with prostatic material as a result of the contact. The collection can be accomplished using the indwelling device that infused the wash fluid into the prostatic portion of the urethra having a lumen in the device that accesses the prostatic portion of the urethra and which is used to withdraw the fluid from that prostatic portion and presumably also at least a portion of one or more of the secretory ducts that feed into the prostatic portion of the urethra. Suction may be applied to the lumen in order to withdraw the fluid if necessary and effective As discussed above, the method can include filling one or more secretory ducts with the washing fluid. This may be accomplished, for example, by filling the prostatic portion of the urethra with washing fluid and continuing to introduce fluid after that portion is filled, forcing the fluid to enter and fill one or more secretory ducts of the prostate. A plurality of the 12-20 secretory ducts can be filled by the method. The terminal vesicles lead from the secretory ducts, and thus washing fluid in the secretory ducts can likely be introduced into the terminal vesicles. Thus the method also comprises having the washing fluid in one or more secretory ducts contact the terminal vesicles. In an additional embodiment, a plurality of terminal vesicles is contacted with washing fluid. In another embodiment, a plurality of terminal vesicles is filled with washing fluid. In a further embodiment, all the secretory ducts and all the terminal vesicles connected to them are filled with washing fluid. The ducts and vesicles are at least partially filled, meaning filled in a range from about 5% any where up to about 100% capacity. This may be accomplished by introducing fluid until backpressure is met. Additionally or alternatively, the prostate can be palpated rectally, to massage the gland and provide fluid access to secretory ducts and terminal vesicles in addition to the fluid that may already have been introduced. It is also possible that a first introduction of fluid and then collection followed by a second introduction of fluid provides for a greater amount of fluid to be introduced the second time.

Methods of treating a prostate condition in a patient according to the present invention include locating the prostatic portion of a urethra 14 comprising an opening of one or more prostatic secretory ducts, and introducing a composition comprising a prophylactic or therapeutic agent throughout the prostatic portion of the urethra and into one or more secretory ducts of the prostate which open into the prostatic portion. The composition is suitable in consistency for passage through the urethra 11 into the prostatic portion of the urethra 14 and into the secretory ducts and possibly one or more terminal vesicles. The prophylactic agent is any agent that can prevent or reduce the onset of a condition in the prostate. The therapeutic agent is any agent that can treat an identified condition in the prostate. The condition is any condition that is manifested in the prostate and which has a name and/or can be described. Such conditions can include, e.g. precancer of the prostate (e.g., low-grade or high-grade PIN), cancer of the prostate (e.g., localized carcinoma or invasive carcinoma), benign prostatic hyperplasia or hypertrophy (BPH), lower urinary tract symptoms (LUTS), inflammation of the prostate, bacterial infection, viral infection, and prostatis.

The method can comprise filling one or more secretory ducts with the composition; the composition in one or more secretory ducts can contact one or more terminal vesicles; a plurality of secretory ducts can be filled with the composition; terminal vesicles can be contacted with the composition; a plurality of terminal vesicles can be filled with the composition; and all the secretory ducts and any terminal vesicles connected to those ducts can be at least partly filled with the composition. Partial filling can be in a range from about 5% to about 95% filling of the secretory duct and/or terminal vesicle.

The introduction of the treatment composition into the prostatic portion of the urethra 14, the secretory ducts and the terminal vesicles, can be accomplished by placing the access device 100 retrograde through the urethra 11 until a delivery portion of the device 100 reaches the prostatic portion of the urethra 14. This position can be determined by rectal palpation of the prostate during advance of the distal tip through the urethra. The device 100 can have a single lumen, more than one lumen, or multiple lumens as suits the purpose of the task of delivering the composition.

The composition comprises active prophylactic or therapeutic agents directed to the identified condition in the prostate. For example, the active agent can be a prophylactic agent against an identified precancer, or a therapeutic agent against an identified cancer in the prostate. Prophylactic and therapeutic agents may be in fact the same agents, administered at different stages of an identified disease. In addition, prophylaxis or therapy can comprise combinations of agents in order to achieve the desired efficacy. The patient may also be monitored during a treatment session (by obtaining more prostate secretory duct cells for analysis; or by following a prognostic marker level) in order to better adapt the treatment (e.g., dosage or frequency of administration) to the patient's treatment needs. The conditions that can be treated by introducing the prophylactic or therapeutic agent can include prostate precancer, prostate cancer, inflammation, prostatis, LUTS, BPH, and other conditions of the prostate.

Prophylactic or therapeutic agents in the composition can include, e.g., a prophylactic or therapeutic protein, polypeptide, peptide, small organic molecule, nucleic acid, antibody, antigen, ligand, analog, antagonist, agonist, hormone, growth factor, or inhibitor. For example, the agent can be antagonist or inhibitor of a molecular actor that induces, permits or encourages growth of malignant or premalignant cells. For example, the agent can be an antagonist or inhibitor of a polypeptide or nucleic acid form of endothelin- 1, basis fibroblast growth factor (bFGF), transforming growth factor-beta (TGF-beta), interleukin-6, interleukin-8, insulin-like growth factor (IGF), platelet-derived growth factor (PDGF), interleukin-1, granulocyte-colony stimulating factor (G-CSF), granulocyte macrophage colony stimulating factor, as described in Pirtskhalaishivili and Nelson, Prostate 2000; 44(1):77-87. The agent may also be nitric oxide also as described in Pirtskhalaishivili and Nelson, Prostate 2000; 44(1):77-87. The active agent can be e.g., an antiandrogen, non-steroidal anti-inflammatory drug (NSAID), selenium, retinoids (e.g., 9-cis-retinoic acid), green and black tea polyphenols, curcumin, phenethyl isothiocyanate, sulforaphane, lycopene, indole-3-carbinol (13C), perillyl alcohol, as described in Kelloff et al, Ann NY Acad Sci 1999; 889: 1-13. Brachytherapy agents, e.g., I¹²⁵ or other known radioisotopes. I can comprise the active agent in the composition. A taxane (e.g., paclitaxel and docetaxel) can comprise the active agent in the composition, as described in Vaishampayan et al., Urology 1999; 54 (6A Supp): 22-9. Monoterpenes (e.g., perillyl alcohol or POH), vitamin D analogs, flavones (e.g., flavopiridol), and angiogenesis inhibitors, as described in Ripple and Wilding, Semin. Oncol. 1999; 26(2): 217-26 can comprise one or more of the active agents in the composition. Non-steroidal anti-androgens, flutamide, bicalutamide, and nilutamide as described in Sarosdy, Anticancer Drugs 1999;10(9): 791-6 can comprise one or more of the active agents in the composition. In general, agents that act as an anti-testosterone agent, such as for example, flutamide, leuprolide and the like, can be delivered to the prostatic ducts 85 to slow or arrest the progress of a testosterone dependent cancer or precancer cell-type. In addition, anticancer agents listed in U.S. Pat. No. 5,763,415, to Sukumar are contemplated as active agents for the composition, to the extent they are effective against prostate cancer and prostate precancer. For example, cytotoxic agents disclosed in U.S. Pat. No. 5,763,415, a cytolytic virus, and/or a vector comprising a gene to destroy ductal epithelium can comprise the active agent in the composition introduced to the prostatic secretory ducts. U.S. Pat. No. 5,763,415, is hereby incorporated by reference in its entirety.

In addition to any of these agents, the composition can comprise additional agents in combination with one or more of a first active agent for achieving increased efficacy in a treatment or prophylaxis. For example a pro-drug or growth inhibitor, or an inhibitor of metastasis, or an agent to prolong the activity of another agent, etc. can be combined with another active agent in the composition introduced to the prostatic portion of the urethra and the prostatic secretory ducts. Agents to reduce bacterial infection and/or inflammation may also be introduced in addition or in conjunction with one or more prophylactic and/or therapeutic agents in order to prevent or treat infection or reduce such present or anticipated symptoms (secondary or primary) as inflammation or prostate enlargement, etc.

In addition, in treating a condition in the prostate, agents that are activatable by energy such as for example heat, x-rays, ultrasound, infrared light, and other light, can be delivered to one or more prostatic ducts 85 and once delivered they can be activated. The energy source can be placed external to the body, or in the rectum or urethra to gain better exposure to the prostate region being treated. Energy conductive solutions for delivery to the prostate are also contemplated.

A radioactive solution can also be delivered to the prostate using the methods of treatment and/or a device of the invention. All isotopes may be effective, particularly those in present usage including e.g. I¹²⁵ seeds or other radioactive solution of iodine.

The composition can also comprise polymers, including those that gel in the body, that can be delivered to the prostate for treatment purposes. Such polymers are well known in the art. These polymers are also capable of providing a controlled time release of the agent to the prostate. The polymers may comprise active therapeutic agents or may carry active therapeutic agents to the site of delivery in the prostate. Colloid solutions may be delivered to the prostate.

Delivery to the prostate can be aided by firstly or concurrently (with delivery of an active agent) delivering a solution to the prostatic ducts 85 that is visible under ultrasound, X-ray or magnetic resonance imaging technologies so that it can be detected whether the ducts have been accessed by the fluid and/or filled, in order to optimize the therapeutic delivery and corresponding effects of the therapeutic agent at the prostate.

A system for treating a prostate condition can comprise the device 100 for accessing the urethra 11 and for retrograde penetration of the urethra 11 to the prostatic portion of the urethra 14 and introduction of a composition to that prostatic portion 14 and the secretory ducts that open to it. Instructions for delivering a composition using the device 100 (describing the methods herein for access and delivery of a composition comprising an active prophylactic or therapeutic agent) and a container for the system contents can also be included. The system can further comprise a composition comprising a prophylactic or therapeutic agent for treating the condition such as those described herein, and or additional adjunctive or palliative agents as also described herein.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for collecting prostatic material comprising:
(a) introducing a washing fluid to a prostatic portion of a urethra; and
(b) collecting at least a portion of the washing fluid comprising prostatic material.

2. A method as in claim 1, wherein the material comprises molecular species.

3. A method as in claim 2, wherein the molecular species comprises one or more of the following: a protein, a polypeptide, a peptide, a nucleic acid, a polynucleotide, an MRNA, a small organic molecule, a lipid, a phospholipid, a fat, a glycoprotein, a glycopeptide, a carbohydrate, an oligosaccharide, a chromosomal abnormality, a particle, a secreted molecule, a cell surface molecule, an intracellular molecule, and a complex of a plurality of molecules.

4. A method as in claim 1, wherein the material comprises cells.

5. A method as in claim 4, wherein the cells comprise prostatic ductal epithelial cells.

6. A method for collecting material from one or more prostatic ducts of a patient said method comprising:
(a) introducing a device through a urethra of a patient,
(b) locating the device in or near a prostatic portion of said urethra,
(c) introducing a washing fluid throughout the prostatic portion, and
(d) collecting at least a portion of the washing fluid mixed with prostatic material.

7. A method as in claim 6, wherein the prostatic material comprises molecular species.

8. A method as in claim 7, wherein the molecular species comprises one or more of the following: a protein, a polypeptide, a peptide, a nucleic acid, a polynucleotide, an mRNA, a small organic molecule, a lipid, a phospholipid, a fat, a glycoprotein, a glycopeptide, a carbohydrate, an oligosaccharide, a chromosomal abnormality, a particle, a secreted molecule, a cell surface molecule, an intracellular molecule, and a complex of a plurality of molecules.

9. A method as in claim 7, wherein the prostatic material comprises cells.

10. A method as in claim 9, wherein the cells comprises prostatic ductal epithelial cells.

11. A method of treating a prostate condition in a patient comprising the steps of:
(a) locating a prostatic portion of a urethra, and
(b) introducing a composition comprising a prophylactic or therapeutic agent to said prostatic portion.

12. A method of treating a prostate condition in a patient comprising:
(a) accessing a prostatic portion of a urethra with an access device, and
(b) introducing a composition comprising a prophylactic or therapeutic agent to said prostatic portion.

13. A method as in claim 12, wherein introduction into the prostatic portion of the urethra comprises also introducing the agent into one or more prostatic ducts.

14. A method as in claim 12, wherein the composition comprises a polymer for facilitating time-release of the prophylactic or therapeutic agent to the prostatic portion.

15. A method as in claim 14, wherein the polymer forms a gel once introduced into the prostatic portion of the urethra.

16. A method as in claim 12, wherein the composition comprises an agent that is activated by an energy source.

17. A method as in claim 14, wherein the energy source is selected from the group consisting of heat, X-rays, ultrasound, infrared light, and other light.

18. A method as in claim 12, wherein the agent comprises a radioactive isotope.

19. A method as in claim 12, wherein the composition can be visualized in the prostatic portion of the urethra by an imaging modality.

20. A method as in claim 17, wherein the imaging modality is selected from the group consisting of ultrasound, X-ray, and magnetic resonance.

21. A method as in claim 12, wherein the agent comprises an anti-testosterone agent.

22. A method as in claim 19, wherein the anti-testosterone agent is selected from the group consisting of flutamide and leuprolide.

23. A device to access a prostatic portion of a urethra comprising an access lumen to infuse wash fluid into the prostatic portion of the urethra and collect prostatic material mixed with wash fluid from within the prostatic portion of the urethra.

24. A device for accessing a prostatic portion of a urethra, said device comprising at least one lumen, a first expandable positioning member, a second expandable positioning member and a fluid delivery portion located between said expandable positioning members for being located proximate prostatic ducts within the urethra, said fluid delivery portion including at least one port for infusing fluid into at least one of the prostatic ducts after said expandable positioning members have been expanded.

25. The device according to claim 24 wherein said expandable positioning members comprise inflatable members.

26. The device according to claim 24 wherein said fluid delivery portion includes a plurality of fluid delivery ports.

27. The device of claim 26 wherein said fluid delivery ports are spaced from each other along the length of the device and around an outer circumference of the lumen.

28. A device for accessing a prostatic portion of a urethra, said device comprising at least one lumen and an expandable fluid delivery portion for being positioned proximate prostatic ducts within the urethra, said fluid delivery portion including at least one port for infusing fluid into at least one of the prostatic ducts after being expanded.

29. The device of claim 28 wherein said expandable delivery portion extends along the length of said at least one lumen.

30. The device of claim 29 wherein said expandable fluid delivery portion comprises a shape memory material.

31. A device for accessing a prostatic portion of a urethra, said device comprising at least one lumen having a diameter that tapers over at least a portion of its length in a direction of a distal end such that a fluid delivery portion of the device forms a tight fit with a inner wall of the urethra carrying a plurality of prostatic ducts, said fluid delivery portion including at least one fluid delivery port for infusing fluid into at least one of the prostatic ducts.

32. The device of claim 31 wherein said fluid delivery portion includes a plurality of fluid delivery ports.

33. A device for accessing a prostatic portion of a urethra, said device comprising at least one lumen having a flexible distal end including an infusion port, a fiberoptic scope and a guidewire formed of a shape memory material that changes an orientation of said flexible distal end when a stimulus is applied to said guidewire.

34. The device of claim 33 wherein said shape memory material includes nitinol.

35. The device of claim 33 wherein said guidewire is positioned within a lumen of said device.

36. The device of claim 33 wherein said fiberoptic scope is positioned along an exterior surface of the at least one lumen.
